(19) United States
(12) Patent Application Publication
(10) Pub. No.: US 2024/0397954 A1
(43) Pub. Date: Dec. 5, 2024

(54) Deodorizing Composition Including Hydrogen Peroxide and Silver Ions

(71) Applicant: Applied Silver, Inc., Haywad, CA (US)

(72) Inventors: Sean Morham, Hayward, CA (US); William Morris, Hayward, CA (US)

(21) Appl. No.: 18/680,149

(22) Filed: May 31, 2024

Related U.S. Application Data
(60) Provisional application No. 63/469,971, filed on May 31, 2023.

(51) Int. Cl. A01N 59/16; A01N 59/00; A01P 1/00; A61L 2/18; A61L 2/238; A61L 101/02

(52) U.S. Cl. CPC A01N 59/16; A01N 59/00; A01P 1/00; A61L 2/186; A61L 2/238; A61L 2101/02; A61L 2202/26

(57) ABSTRACT

The present disclosure relates generally to a deodorizing composition including hydrogen peroxide, present in an amount of no more than 3 wt. %, based on the total weight of the composition; silver ions, present in an amount in the range of 1-1000 ppm, based on the total weight of the composition; and water, present in an amount of at least 90 wt. %, based on the total weight of the composition.

### CROSS-REFERENCE TO RELATED APPLICATIONS

This application claims the benefit of U.S. Provisional Patent Application No. 63/469,971, filed May 31, 2023, which is hereby incorporated herein by reference in its entirety.

### FIELD

The present disclosure relates generally to a deodorizing composition that includes hydrogen peroxide and silver ions for effective elimination of odors-causing microbes, residues, and germs.

### TECHNICAL BACKGROUND

Unpleasant smells, caused by various microbes, residues, and germs, can be present in any number of environments, including homes, workplaces, and public spaces. In particular, odor-causing microbes can often linger on soft surfaces, such as fabrics or linens. These soft surface materials may be used in clothing, bedsheets, towels, pillows, blankets, and similar materials. Traditional products used to mitigate unwanted odors include air fresheners, masking agents, and deodorizers.

However, many of these products simply mask the unwanted odors instead of eliminating the cause of the odor itself. Additionally, these products often contain chemicals that can be harmful to the environment and human health, particularly if used on the soft surface materials above, some or all of which may make frequent and prolonged contact with a human's skin. As such, there is a need in the art to provide products and methods that can eliminate odors, and the microbes, residues, and germs that cause them, in an efficient and safe way and be particularly safe for human contact and use.

### SUMMARY

In one aspect, the present disclosure provides methods of deodorizing, the methods including applying the deodorizing composition, as described herein, to the source of an odor. In particular aspects of the present disclosure, the deodorizing composition is applied to soft surfaces, such as textiles.

In another aspect, the present disclosure provides a deodorizing composition including:
hydrogen peroxide, present in an amount of no more than 3 wt. %, based on the total weight of the composition;
silver ions, present in an amount in the range of 1-1000 ppm, based on the total weight of the composition; and
water, present in an amount of at least 90 wt. %, based on the total weight of the composition.

In another aspect, the present disclosures provides a deodorizing composition including:
hydrogen peroxide, present in an amount of no more than 3 wt. %, based on the total weight of the composition;
silver ions, present in an amount in the range of 1-1000 ppm, based on the total weight of the composition;
a fragrance, present in an amount of at least 0.1 wt. %, based on the total weight of the composition; and
water, present in an amount of at least 90 wt. %, based on the total weight of the composition.

In another aspect, the present disclosure provides a deodorizing composition including:
hydrogen peroxide, present in an amount of 1.5 wt. %, based on the total weight of the composition;
silver ions, present in an amount of 25 ppm, based on the total weight of the composition; and
water, present in an amount of at least 97 wt. %, based on the total weight of the composition.

In another aspect, the present disclosure provides a deodorizing composition including:
hydrogen peroxide, present in an amount of 1.5 wt. %, based on the total weight of the composition;
silver ions, present in an amount of 25 ppm, based on the total weight of the composition;
a fragrance, present in an amount of 0.5 wt. %, based on the total weight of the composition; and
water, present in an amount of at least 97 wt. %, based on the total weight of the composition.

In another aspect, the present disclosure provides a method for treating a textile with a deodorizing composition, the method including applying the deodorizing composition to the textile, wherein the deodorizing composition comprises any of the deodorizing compositions as described herein.

### BRIEF DESCRIPTION OF FIGURES

The accompanying drawings are included to provide a further understanding of the methods of the disclosure, and are incorporated in and constitute a part of this specification. The drawings are not necessarily to scale, and sizes of various elements may be distorted for clarity. The drawings illustrate one or more embodiment(s) of the disclosure and together with the description serve to explain the principles and operation of the disclosure.

FIG. 1 is a picture of colored and uncolored textiles treated with an example composition as described herein.

FIG. 2A is a picture of an uncolored textile after multiple treatments with an example composition as described herein.

FIG. 2B is a picture of uncolored napkin and uncolored texted after multiple treatments with a comparative composition.

FIG. 3 is a picture of a human foot directly sprayed with an example composition as described herein.

FIG. 4 is a flow chart illustrating an example method as described herein.

### DETAILED DESCRIPTION

The present disclosure provides example methods and compositions (including a deodorizing composition) that effectively eliminates odor-causing microbes, residues, and germs. As described above, current products used to remove unwanted odors often do not remove the cause of the odor, but simply mask the scent. Further, these products often contain harsh chemicals that can be harmful to the environment and human health. The present disclosure is concerned with compositions that are useful for deodorizing, cleaning, cleansing, and effectively eliminating the cause of malodor. As such, in one aspect, the present disclosure provides a deodorizing composition including hydrogen peroxide and silver ions. In one embodiment as described herein, the present disclosure provides a deodorizing composition including hydrogen peroxide, present in an amount of no more than 3 wt. %, based on the total weight of the composition; silver ions, present in an amount in the range of 10-1000 ppm, based on the total weight of the composition; and water, present in an amount of at least 90 wt. %, based on the total weight of the composition.

In one embodiment, an example deodorizing composition is described herein that effectively eliminates odors while also being safe for use in a household environment. In a further aspect, the example composition is also easy to use and does not leave behind harmful residues. In one example embodiment, advantageously, the combination of hydrogen peroxide and silver ions effectively eliminates odors and odor-causing microbes better than compositions that include these components individually. Additionally, in examples, the odor-eliminating properties of the composition are observed at relatively low concentrations of both hydrogen peroxide and silver ions. In a further aspect, in example embodiments, the oxidizing action of hydrogen peroxide and the binding action of silver ions work together to stop any incidental odors created by either ingredient on its own. Further, in example embodiments, the successful odor elimination can be accomplished at low concentrations of each components due to the synergistic relationship observed between the hydrogen peroxide and silver ions. As such, one example deodorizing composition, as described herein, provides a dual-action mechanism to effectively eliminate odors based on the synergy between hydrogen peroxide and silver ions at concentrations safe for household use.

As described above, the deodorizing composition as described herein includes hydrogen peroxide. Hydrogen peroxide is a powerful oxidizing agent that can break down and destroy the source of the odor. It also has antimicrobial properties, making it effective against odor-causing microbes (i.e., bacteria, molds, yeasts, etc.). However, hydrogen peroxide can also adversely react with and cause discoloration of surfaces it is disposed on. For example, hydrogen peroxide is known to bleach textiles. As such, the amount of hydrogen peroxide in the composition as described herein is balanced to provide effective antimicrobial efficacy, but without adverse effects (e.g., discoloring textiles). As described above, in some embodiments, hydrogen peroxide is present in an amount of no more than 3 wt. %, based on the total weight of the composition. For example, in some embodiments, hydrogen peroxide is present in an amount of no more than 2.5 wt. %, or 2 wt. %, or 1.5 wt. %, based on the total weight of the composition. In various embodiments as described herein, hydrogen peroxide is present in a range of 0.1 to 3 wt. %, or 0.1 to 2.5 wt. %, or 0.1 to 2 wt. %, or 0.1 to 1.5 wt. %, or 0.5 to 3 wt. %, or 0.5 to 2.5 wt. %, or 0.5 to 2 wt. %, or 0.5 to 1.5 wt. %, or 1 to 3 wt. %, or 1 to 2.5 wt. %, or 1 to 2 wt. %, or 1 to 1.5 wt. %, based on the total weight of the composition. In particular embodiments of the present disclosure as described herein, hydrogen peroxide is present in an amount of 1.5 wt. %, based on the total weight of the composition. The amounts of hydrogen peroxide as described herein have been found to be optimal for both deodorizing and disinfecting purpose, while also being safe for use in a household environment.

The deodorizing composition as described herein also includes silver ions. Silver ions are effective at neutralizing odors and killing odor-causing microbes. However, as with hydrogen peroxide, silver ions can also have adverse effects when used in high quantities. As such, the amount of silver ions in the composition as described herein is balanced to provide effective antimicrobial efficacy, but without adverse effects. In some embodiments, silver ions are present in an amount in the range of 1 to 1000 ppm, based on the total weight of the composition. For example, in various embodiments, silver ions are present in an amount in the range of 1 to 500 ppm, or 1 to 100 ppm, or 1 to 50 ppm, or 1 to 25 ppm, or 10 to 1000 ppm, or 10 to 500 ppm, or 10 to 100 ppm, or 10 to 50 ppm, or 10 to 25 ppm, or 15 to 1000 ppm, or 15 to 500 ppm, or 15 to 100 ppm, or 15 to 50 ppm, or 15 to 15 ppm, or 20 to 1000 ppm, or 20 to 500 ppm, or 20 to 100 ppm, or 20 to 50 ppm, or 20 to 25 ppm, based on the total weight of the composition. In particular embodiments of the present disclosure as described herein, silver ions are present in an amount of 25 ppm. The amounts of silver ions as described herein have been found to be sufficient to provide odor-eliminating effects, while minimizing the amount of silver ion used. These concentrations are safe for household use.

A silver compound is used to provide the silver ions to the composition. In some embodiments, the silver compound is water-soluble. For example, in some embodiments, the silver compound is a silver salt. The silver salt may be selected from silver nitrate, silver sulfate, silver fluoride, silver carbonate, silver chloride, silver acetate, or any combination thereof. The silver compound may be present in an amount sufficient to provide the amount of silver ions as described herein to the composition. In some embodiments as described herein, the silver ions are provided in the form of a silver salt. For example, the silver ions may be provided as silver nitrate, silver sulfate, silver fluoride, silver carbonate, silver chloride, silver acetate, or any combination thereof.

In some embodiments of the present disclosure as described herein, the hydrogen peroxide and silver ions are provided in a weight ratio of no more than 3,000:1. For example, in some embodiments, the hydrogen peroxide and silver ions are provided in a weight ratio of no more than 2,000:1, or 1,000:1, or 800:1, or 600:1. In various embodiments as described herein, the weight ratio of hydrogen peroxide to silver ions is in the range of 1:1 to 3,000:1, or 200:1 to 3,000:1, or 500:1 to 3,000:1, or 1:1 to 2,000:1, or 200:1 to 2,000:1, or 500:1 to 2,000:1, or 1:1 to 1,000:1, or 200:1 to 1,000:1, or 500:1 to 1,000:1. In some embodiment as described herein, the weight ratio of hydrogen peroxide to silver ions is in the range 500:1 to 800:1, or 500:1 to 700:1, or 500:1 to 600:1. In some embodiments, the weight ratio of hydrogen peroxide to silver ions is 600:1.

In another aspect, the present disclosures provides a deodorizing composition including hydrogen peroxide, present in an amount of no more than 3 wt. %, based on the total weight of the composition; silver ions, present in an amount in the range of 10-1000 ppm, based on the total weight of the composition; a fragrance, present in an amount of at least 0.1 wt. %, based on the total weight of the composition; and water, present in an amount of at least 90 wt. %, based on the total weight of the composition.

The fragrance is not particularly limited and may be selected from any fragrance known in the art. In some embodiments as described herein, the fragrance is a bio-identical fragrance. As used herein, a bio-identical fragrance is a fragrance made in a laboratory with the same chemical structure as a naturally occurring fragrance. As described above, the fragrance may be present in an amount of at least 0.1 wt. %, based on the total weight of the composition. For example, in some embodiments, the fragrance may be present in an amount of at least 0.2 wt. %, or at least 0.5 wt. %, based on the total weight of the composition. In various embodiments, the fragrance is present in an amount in the range of 0.1 to 2 wt. %, or 0.1 to 1.5 wt. %, or 0.1 to 0.5 wt. %, or 0.2 to 2 wt. %, or 0.2 to 1.5 wt. %, or 0.2 to 0.5 wt. %, based on the total weight of the composition. In particular embodiments as described herein, the fragrance is present in an amount of 0.5 wt. %, based on the total weight of the composition. In particular embodiments as described herein, the fragrance is present in an amount of 0.3 wt %, based on the total weight of the composition.

In various embodiments as described herein, the deodorizing composition may further comprise a surfactant. It has been found that including a surfactant in the deodorizing composition can provide improved cleaning and deodorizing power by providing improved access and miscibility of the hydrogen peroxide and silver ions with odorous molecules. In various embodiments as described herein, the surfactant is a non-ionic surfactant. For example, in some embodiments, the surfactant is selected from one or more of an alcohol ethoxylate (e.g., a fatty alcohol ethoxylate), an alkyl phenol ethoxylate, and an acid alkoxylate (e.g., a fatty acid alkoxylate). In particular embodiments as described herein, the surfactant is an alcohol ethoxylate, e.g., a fatty alcohol ethoxylate. Examples of non-ionic surfactants suitable for the deodorizing composition described herein are sold under the designations BIO-SOFT®N-900, available from Stepan Company, or are sold under the designations ECOSURF™ EH-9 Surfactant, available from Dow, or are sold under the designations Norfox® 609 NR, available from Norman, Fox & Co. The surfactant may be present in an amount of no more than 5 wt. %, based on the total weight of the composition. For example, in some embodiment, the surfactant is present in an amount of no more than 3 wt. % or no more than 2 wt. %. In various embodiments as described herein, the surfactant is present in an amount in the range of 0.1 to 5 wt. %, 0.1 to 3 wt. %, or 0.1 to 2 wt. %, or 0.2 to 5 wt. %, or 0.2 to 3 wt. %, or 0.2 to 2 wt. %, or 0.5 to 5 wt. %, 0.5 to 3 wt. %, or 0.5 to 2 wt. %, based on the total weight of the composition

The deodorizing composition as described herein may further include one or more additional additives. For example, such additional additives may be selected from stabilizing agents, pH modifiers, chelating agents, viscosity modifiers, preservatives, and antibacterial compounds. The additional additives may be present in the deodorizing composition in various amounts. For example, the one or more additional additives may be present in an amount in the range of 0.1 to 5 wt. %, or 0.1 to 2 wt. %, or 0.1 to 1 wt. %, or 0.2 to 5 wt. %, or 0.2 to 2 wt. %, or 0.2 to 1 wt. %, based on the total weight of the composition.

As would be understood by the person of ordinary skill in the art, many silver compounds, such as silver nitrate, and solutions made therefrom can be light sensitive. Advantageously, the present disclosure provides a deodorizing composition that includes silver ions that is stable in light. Accordingly, the deodorizing composition as described herein does not include a photo-stabilizing agent.

As described above, the deodorizing composition as disclosed herein also includes water. Water may be present in various amount sufficient to bring the total amount of the composition to 100%. For example, in various embodiments, water is present in an amount of at least 90 wt. %, or at least 95 wt. %, or at least 97 wt. %, based on the total weight of the composition. In some embodiments as described herein, water is present in an amount of at least 97 wt. %, based on the total weight of the composition. In some embodiments as described herein, the water is deionized water (i.e. DI water).

In various embodiments, the deodorizing composition of the disclosure as described herein has a pH in the range of 5-8. For example, the deodorizing composition may have a pH in the range of 5.5-8, or 5.5-7.5, or 6-8, or 6-7.5, or 7-8, or 7-7.5. Accordingly, the deodorizing composition of the disclosure does not include a substantial amount of water-soluble alkaline compounds (i.e., alkali or alkaline-earth metal hydroxides or water-soluble alkali or alkaline-earth metal salts). For example, in various embodiments as described herein, the deodorizing composition of the disclosure as described herein includes less than 5 wt. %, e.g., less than 3 wt. %, or less than 2 wt. %, or less than 1 wt. %, of water-soluble alkaline compounds.

As described above, the deodorizing composition includes water in various amounts to bring the total amount of the composition to 100%. Accordingly, the deodorizing composition as described herein is an aqueous solution. As used herein, an aqueous solution includes a substantial amount (e.g., at least 90 wt. %, at least 95 wt. %, or at least 97 wt. %) of water. In some embodiments as described herein, the deodorizing composition does not include an oil phase (e.g., an organic phase). For example, in some embodiments as described herein, the deodorizing composition is not an emulsion (e.g., not an oil-in-water emulsion).

As would be understood by the person of ordinary skill in the art, hydrogen peroxide can react with many silver compounds to provide various products such as silver peroxide, oxygen gas, water, or inorganic acid (e.g., when silver nitrate is used, nitric acid may be formed). Additionally, the person of ordinary skill in the art would appreciate that silver peroxide has a dark (e.g., brown to black) appearance and is insoluble in water. Advantageously, the present disclosure includes low amounts of both hydrogen peroxide and silver ions to avoid substantial amounts of the reaction products of the two components and any color that may result from the reaction product. As such, the deodorizing composition of the present disclosure is a colorless aqueous solution (e.g., a clear aqueous solution).

As described above, the deodorizing composition as described herein is an aqueous solution. As such, the deodorizing composition may be easily applied to the source of an odor. For example, in some embodiments, the composition is in the form of a spray.

As described above, another aspect of the present disclosure provides a method of deodorizing. The method includes applying the deodorizing composition to the source of an odor. The source of the odor is not particularly limited. For example, the source of the odor may originate from household items, waste, or be present in the air. Common examples of sources of odor include trash, garbage cans, fabrics, or litterboxes. In some embodiments as described herein, the deodorizing composition may be applied directly to the source of the odor. In other embodiments, the deodorizing composition may be applied indirectly to the source of the odor. For example, in some embodiments, the deodorizing composition may be applied to the air or space that includes an odor. In some embodiments as described herein, applying the deodorizing composition includes spraying the composition. As such, in some embodiments, the deodorizing composition may be used to freshen up a room by spraying the composition into the air.

As described above, another aspect of the present disclosure provides a method of deodorizing surfaces by applying the deodorizing composition, as described herein, onto the surface, e.g., hard and/or soft surface. Such hard and soft surfaces are not particularly limited. For example, they may include plastics, wood, granite, quartz, and other minerals, metals, textiles, fabrics, and clays. In particular aspects of the present disclosure, the deodorizing composition is sprayed onto the surface. For example, in some embodiments, the deodorizing composition is sprayed as a fine mist. In various embodiments as described herein, the fine mist has an average droplet size of less than 50 μm, or less than 25 μm, or less than 10 μm. In some embodiments as described herein, the deodorizing composition is applied at a rate of at least 0.1 mL/second, or at least 0.5 mL/second, or at least 1 mL/second. In various embodiments as described herein, the deodorizing composition is applied at a rate in the range of 0.2 to 2 mL/second, or 0.5 to 2 mL/second, or 1 to 2 mL/second, or 0.2 to 1.5 mL/second, or 0.5 to 1.5 mL/second, or 1 to 1.5 mL/second. To accomplish the application of the deodorizing compositions in a fine mist, one or more of several mechanisms may be employed, including: (i) an aerosol container; (ii) one or more fans; (iii) a particularly configured nozzle (e.g., a nozzle with one or more interior grooves of one or more orifice cups); and/or (iv) an actuator.

In particular aspects, the surfaces described herein are textiles. As such, another aspect of the present disclosure provides a method of deodorizing textiles by applying the deodorizing composition, as described herein, onto the textile. The textile is not particularly limited. For example, the textile may include cotton, polyester, spandex, natural fibers, synthetic fibers, or combinations thereof. In particular aspects of the present disclosure, the deodorizing composition is sprayed, as described herein, onto the textile.

The deodorizing composition and methods as described herein remove or eliminate unpleasant smells and odors. Malodor may be caused by a variety of factors and may be from a variety of sources. The cause of the malodor is not particularly limited. The deodorizing composition and methods as described herein remove or eliminate the cause of malodors. For example, in some embodiments, the deodorizing composition and methods as described herein remove or eliminate odor-causing microbes, residues, and germs.

### EXAMPLES

The Examples that follow are illustrative of specific embodiments of the composition of the disclosure and various uses thereof. They are set forth for explanatory purposes only and are not to be taken as limiting the scope of the disclosure.

### Example 1. General Cleaning Capabilities

The components of the composition as described herein where tested separately and together at varying concentrations for their anti-odor and general cleaning capabilities. Three different concentrations for silver ions (10 ppm, 25 ppm, and 30 ppm) and two different concentrations of hydrogen peroxide (1.5 wt % and 3 wt %) were used. These tests included compositions with and without a fragrance. In general, compositions with both silver ions and hydrogen peroxide, and optionally a fragrance, were found to have cleaning capabilities.

### Example 2. Stain Testing

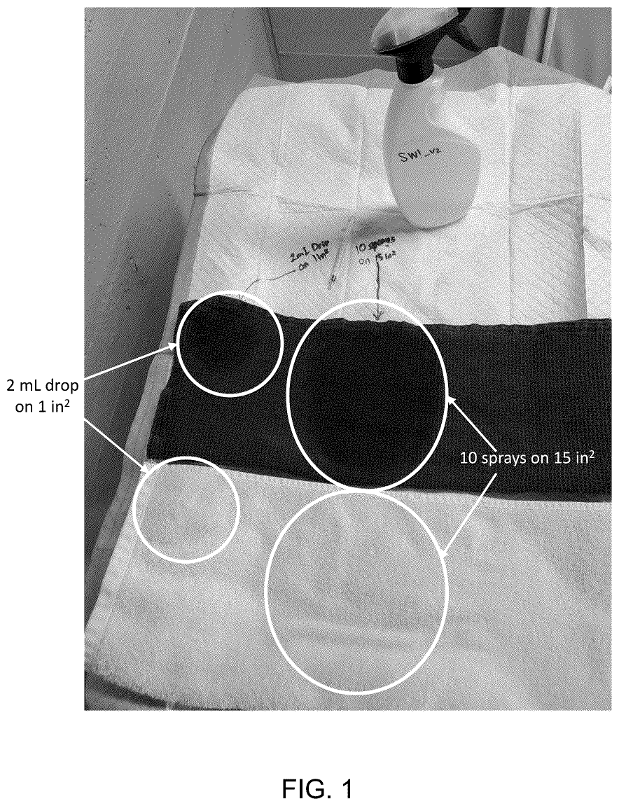

Stain-testing was performed on white and black textiles (fabrics) to determine if any visible damage (e.g., discoloration) was observed upon application of the compositions as described herein. The composition used included 1.5 wt % hydrogen peroxide, 25 ppm silver ions, with the remainder being water. On both a colored (black) and uncolored (white) fabric, 2 mL of the composition was applied on a 1 in² area and 10 sprays of the composition from a spray nozzle bottle was sprayed on a 15 in² area. After application of the composition, each fabric was allowed to dry for a period of at least 3 hours. FIG. 1 is a picture of the colored (black) and uncolored (white) fabrics treated with the composition. No visible bleaching or staining was observed on either fabric tested.

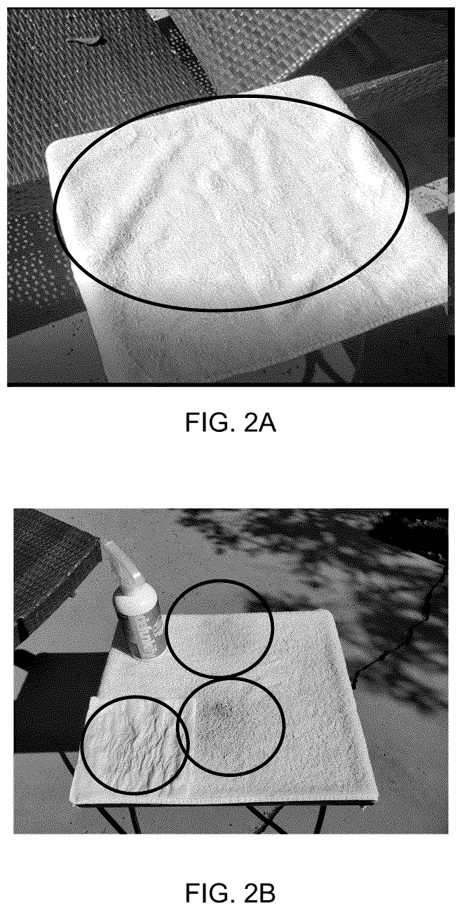

Additionally, on an uncolored (white) fabric, two sprays of the composition from a spray nozzle bottle was applied on approximately a 15 in² area. After application of the composition, the fabric was allowed to dry for a period of at least 3 hours. This application was repeated 5 times. FIG. 2A is a picture of the uncolored fabric after 5 applications of the composition. No visible bleaching or staining is observed on the uncolored fabric tested. A comparative composition was also tested by applying two sprays of the comparative composition from a spray nozzle bottle on an uncolored (white) napkin (e.g., paper based product) and an uncolored (white) fabric. This application was repeated 5 times. The comparative composition was an odor-reducing spaying including 20 ppm ionic silver and essential oils. FIG. 2B is a picture of the uncolored (white) napkin and fabric. In contrast to FIG. 2A, the comparative product causes discoloration of both the uncolored napkin and uncolored fabric.

Additional stain-testing was performed on uncolored (white) fabrics to determine if the compositions as described herein can lead to discoloration. As would be understood by the person of ordinary skill in the art, silver is photosensitive and so this test was performed while the fabric was exposed to direct sunlight. For each test, 10 re-applications of the composition was applied on white fabric. The composition used included 1.5 wt % hydrogen peroxide, 25 ppm silver ions, with the remainder being water. Two comparative compositions, including 25 ppm silver ions and 20 ppm silver ions respectively, were also applied to the uncolored fabric. After application of the composition, each fabric was allowed to dry for a period of at least 6 hours. No discoloration was observed for the composition including both hydrogen peroxide and silver.

### Example 3. Anti-Odor Efficacy for Soft Surfaces

The composition including 1.5 wt % hydrogen peroxide, 25 ppm silver ions, with the remainder being water was evaluated for its anti-odor efficacy. The composition was applied to a number of different soft surface that made contact with human skin. In a first experiment, the composition was applied to shoes and sock that were worn together on a human foot for 3 days. The odor of the socks and shoes were reduced after a single application of the compositions. No deleterious effects (e.g., corrosivity or discoloration) were observed on any human skin surface that contacted the treated shoe, the treated sock, or the combination of the two. In a second experiment, the composition was also tested on pillows and upholstery and was found to decrease the odor. No discoloration of the textiles or deleterious effects (e.g., corrosivity or discoloration) on any human skin surface that contacted the treated textiles were observed.

### Example 4. Skin Corrosivity

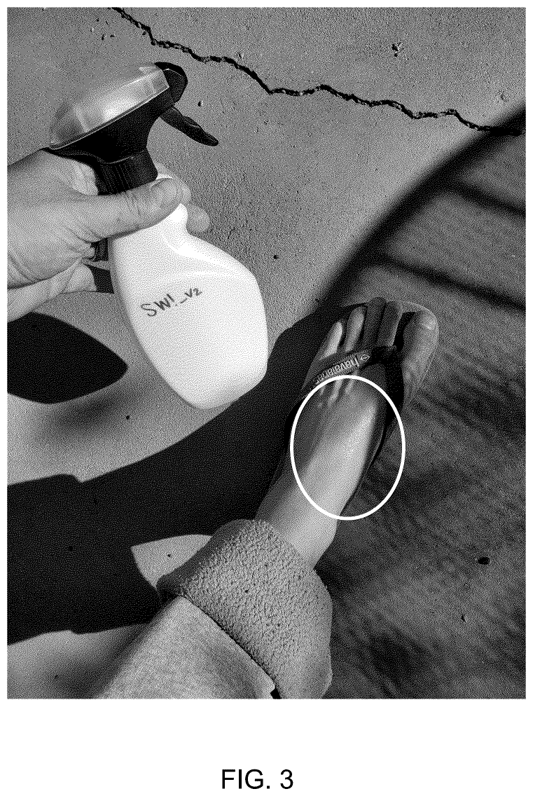

The composition including 1.5 wt % hydrogen peroxide, 25 ppm silver ions, with the remainder being water was evaluated for its skin corrosivity. The composition was sprayed directly on a human foot and allowed to air-dry without any rinsing. The application was not rinsed or wiped off for at least 24 hours and in that time no noticeable changes or symptoms were observed. FIG. 3 is a picture of the foot after treatment with the composition. As indicated in FIG. 3, no deleterious effects (e.g., corrosivity or discoloration) were observed on the foot after spraying the composition directly on the foot.

### Example Method

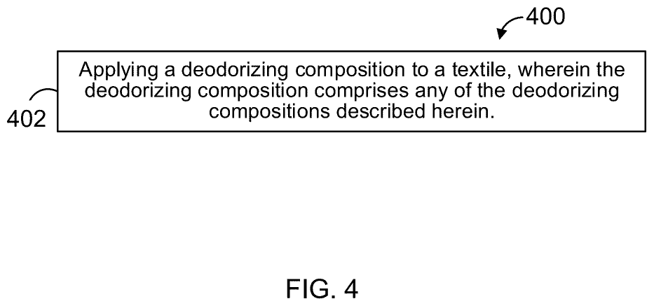

FIG. 4 is a flow chart illustrating an example method 400.

At block 402, the method 400 can include, treating a textile with a deodorizing composition, the method comprising applying the deodorizing composition to the textile, wherein the deodorizing composition comprises any of the deodorizing compositions detailed herein. In some examples, applying the deodorizing composition to the textile comprises spraying the deodorizing composition on the textile. In other examples, applying the deodorizing composition to the textile comprises submerging the textile in the deodorizing composition.

In still other examples, applying the deodorizing composition to the textile comprises washing the textile in a wash basin in the presence of any of the deodorizing compositions detailed herein. In a further aspect, in example embodiments, method 400 further comprises transferring the treated textile from the wash basin to a dryer and drying the treated textile. In examples, after drying the treated textile, the treated textile maintains a predetermined antimicrobial efficacy.

### Example Variations

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings and/or examples making apparent to those skilled in the art how the several forms of the disclosure may be embodied in practice. Thus, before the disclosed processes and devices are described, it is to be understood that the aspects described herein are not limited to specific embodiments, apparatuses, or configurations, and as such can, of course, vary. It is also to be understood that the terminology used herein is for the purpose of describing particular aspects only and, unless specifically defined herein, is not intended to be limiting.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. It will be further understood that the endpoints of each of the ranges are significant both in relation to the other endpoint, and independently of the other endpoint.

All methods described herein can be performed in any suitable order of steps unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As will be understood by one of ordinary skill in the art, each embodiment disclosed herein can comprise, consist essentially of or consist of its particular stated element, step, ingredient or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment.

Unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Some embodiments of this disclosure are described herein, including the best mode known to for carrying out the disclosure. Of course, variations on these described embodiments will become apparent to those of ordinary skill in the art upon reading the foregoing description. The disclosure allows skilled artisans to employ such variations as appropriate, and the disclosure is intended to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Furthermore, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

We claim:

1. A deodorizing composition comprising:
hydrogen peroxide, present in an amount of no more than 3 wt. %, based on the total weight of the composition;
silver ions, present in an amount in the range of 1-1000 ppm, based on the total weight of the composition; and
water, present in an amount of at least 90 wt. %, based on the total weight of the composition.

2. The deodorizing composition of claim 1, further comprising a fragrance, present in an amount of at least 0.1 wt. %, based on the total weight of the composition.

3. The deodorizing composition of claim 1, further comprising a surfactant, present in an amount of no more than 3 wt. %, based on the total weight of the composition.

4. The deodorizing composition of claim 1, wherein the hydrogen peroxide is present in an amount in the range of 0.1 to 2.5 wt %, based on the total weight of the composition.

5. The deodorizing composition of claim 1, wherein the silver ions are present in an amount in the range of 1 to 100 ppm, based on the total weight of the composition.

6. The deodorizing composition of claim 1, wherein the silver ions are provided in the form of a silver salt.

7. The deodorizing composition of claim 6, wherein the silver salt is selected from silver nitrate, silver sulfate, silver fluoride, silver carbonate, silver chloride, silver acetate, or any combination thereof.

8. The deodorizing composition of claim 1, wherein the weight ratio of hydrogen peroxide to silver ions is in the range of 1:1 to 3,000:1.

9. The deodorizing composition of claim 1, wherein the water is present in an amount of at least 95 wt. %, based on the total weight of the composition.

10. The deodorizing composition of claim 1 having a pH in the range of 5-8.

11. The deodorizing composition of claim 1, wherein
the hydrogen peroxide is present in an amount of 1.5 wt. %, based on the total weight of the composition;
the silver ions are present in an amount of 25 ppm, based on the total weight of the composition; and
the water is present in an amount of at least 97 wt. %, based on the total weight of the composition.

12. The deodorizing composition of claim 2, wherein
the hydrogen peroxide is present in an amount of 1.5 wt. %, based on the total weight of the composition;
the silver ions are present in an amount of 25 ppm, based on the total weight of the composition;
the fragrance is present in an amount of 0.5 wt. %, based on the total weight of the composition; and
the water is present in an amount of at least 97 wt. %, based on the total weight of the composition.

13. A deodorizing composition comprising:
hydrogen peroxide, present in an amount of no more than 3 wt. %, based on the total weight of the composition;
silver ions, present in an amount in the range of 1-1000 ppm, based on the total weight of the composition;
a fragrance, present in an amount of at least 0.1 wt. %, based on the total weight of the composition;
a surfactant, present in an amount of no more than 3 wt. %, based on the total weight of the composition; and
water, present in an amount of at least 90 wt. %, based on the total weight of the composition.

14. The deodorizing composition of claim 13, wherein the fragrance is a bio-identical fragrance.

15. A method for treating a textile with a deodorizing composition, the method comprising:
applying the deodorizing composition to the textile, wherein the deodorizing composition comprises the deodorizing compositions of claim 1.

16. The method of claim 15, wherein applying the deodorizing composition to the textile comprises spraying the deodorizing composition on the textile.

17. The method of claim 15, wherein applying the deodorizing composition to the textile comprises submerging the textile in the deodorizing composition.

18. The method of claim 15, wherein applying the deodorizing composition to the textile comprises washing the textile in a wash basin in the presence of the deodorizing composition.

19. The method of claim 18, wherein the method further comprises transferring the treated textile from the wash basin to a dryer and drying the treated textile.

20. The method of claim 19, wherein after drying the treated textile, the treated textile maintains a predetermined antimicrobial efficacy.
